(19) United States
(12) Patent Application Publication
(10) Pub. No.: US 2011/0071636 A1
(43) Pub. Date: Mar. 24, 2011

(54) Displacement Leaf Spring and Artificial Intervertebral Disc Containing the Same

(75) Inventors: Yang-Hwei Tsuang, Taipei (TW); Cheng-Kung Cheng, Taipei (TW); Hung-Wen Wei, Taipei (TW); Chang-Jung Chiang, Taipei (TW); Wen-Chuan Chen, Taipei (TW); Yueh-Feng Chiang, Taipei (TW); Wei-Cheng Wang, Taipei (TW)

(73) Assignee: NATIONAL YANG MING UNIVERSITY, Taipei (TW)

(21) Appl. No.: 12/628,133

(22) Filed: Nov. 30, 2009

(30) Foreign Application Priority Data
Sep. 18, 2009 (TW) 098131505

(51) Int. Cl. A61F 2/44

(52) U.S. Cl. 623/17.13

(57) ABSTRACT

A displacement leaf spring and an artificial intervertebral disc containing the same, particularly about the artificial intervertebral disc using the displacement leaf spring to cushion axial compression, wherein the artificial intervertebral disc includes a displacement leaf spring and the displacement leaf spring further includes an upper plate, an inclined plate, and a lower plate. The upper plate has a first breach, and the inclined plate has a first connecting side which connects to one side of the first breach. There is an angle between the inclined plate and the upper plate. The inclined plate has a second breach, and the lower plate has a second connecting side which connects to one side of the second breach. There is an angle between the lower plate and the inclined plate, and the lower plate parallels the upper plate. The artificial intervertebral disc equipped with the displacement leaf spring further comprises a top plate, a bottom plate, a core structure, and a socket structure. The top and bottom plate connect to the bottom of the upper vertebral body and the top of the lower vertebral body respectively. The form of the core structure fits in the socket structure. The core structure and the socket structure are installed between the top and bottom plate to make the artificial intervertebral disc have a wide range of activity angle and capability to cushion the axial compression.

### BACKGROUND OF THE INVENTION

1. Field of the Invention

The present invention relates to the displacement leaf spring and the artificial intervertebral disc containing the same, particularly about the artificial intervertebral disc using the displacement leaf spring to cushion axial compression.

2. Prior Art Introduction

With the growth of the human body age, the organs of the body will slowly degenerate over time. In terms of spine, the intervertebral disc is one of the structures which is the easiest to degenerate over time. The intervertebral disc is between the upper vertebral body and the lower vertebral body to be the main connecting support structure and also the main cushion structure to sustain the axial compression of the spine. When the intervertebral disc degenerates, the lower water content often results in the decreasing of the intervertebral joint's height and the loss of flexibility simultaneously, which brings about compression of nerve roots or spinal cord, and then causes pathological changes. The condition includes pain in partial body of the patient, weakness of extremities, neuralgia, paraesthesia or function disturbance, and the foregoing makes the patient extremely indisposed.

When intervertebral disc of the patient degenerates or with pathological changes, the common surgery is spinal interbody fusion. The principle of spinal interbody fusion is to transplant the cage to the place between the two vertebrae of a patient. The cage is used to restore or maintain intervertebral spacing and avoid the compression of nerve roots, which may cause other diseases to complicate at the same time. Eventually, combine the vertebral body with the artificial intervertebral disc just as the real one to make the structure of vertebral body much firmer. However, spinal interbody fusion will increase the stress around the vertebral body, which leads to compensatory degeneration also known as secondary degeneration and increases the activity of forward tilting, backward tilting, and rotating motility of adjacent joint. Above mention will accelerate the degeneration and pathological changes of adjacent joint, therefore the development of keeping the primary range of movement of the real intervertebral disc is important to retrieve the defect of the spinal interbody fusion.

There are a variety of products of artificial intervertebral discs nowadays; even so, most of the conventional artificial interverbral discs can only provide moderate angular displacement for the upper vertebral body and the lower vertebral body to simulate the structure of the human body, but not like the real interverbral discs having elasticity to cushion the axial compression between two vertebrae. The axial compression between two vertebrae is unavoidable, for example, going upstairs and downstairs, walking, and so forth. The biggest shortcoming of the current artificial intervertebral disc is that most of them do not have axial cushion structure so that the artificial intervertebral discs have to absorb the compression directly and will be damaged early.

In view of the above, several artificial intervertebral discs having axial cushion structure such as U.S. Pat. No. 5,893,889 using an annular shock absorbing member 68 composed of flexible material and a shock absorbing plug 69 to absorb axial shock energy.

Further, as shown in US publication number US2002/0130112A1, U.S. Pat. No. 6,520,996, and U.S. Pat. No. 6,802,867, these patents use the axial support 200 composed of flexible material and plurality of torsional supports 300A, 300B to connect the first plate 100 and the second plate 102, wherein the torsional supports 300A, 300B are used to provide sufficient resistance to bending and torsion to allow the artificial intervertebral disc to support surrounding tissue and prevent injury due to excessive bending or torsion, and the axial support 200 is used to cushion the axial compression between vertebral bodies.

In addition, as shown in US publication number US2004/0073310A1, this patent uses the articulating core member 51 to cushion the axial compression between two vertebrae, and flexible polymer installed around the articulating core member 51 to cushion the torsion between two vertebrae.

Further, as shown in US publication number US2005/0197702A1, this patent is about an artificial intervertebral disc composed of flexible polymer material and shaped into a hollow ring, wherein the artificial intervertebral disc comprising a valve 20, the valve 20 providing access to the interior 19 of the artificial intervertebral disc so that fluid 22 may be injected into, or removed from, the interior 19 of the artificial intervertebral disc to cushion all sorts of compression between two vertebrae.

Furthermore, as shown in US publication number US2005/0228500A1 and US2007/0168033A1, these patents use the fibrous compressible element 17 composed of the fiber 16 to simulate the displacement capability of the real intervertebral disc under the condition of being compressed by placing the fibrous compressible element 17 between two vertebrae.

As shown in US publication number US2005/0251260A1, the characteristic of this patent is using the spring element 30, the elastomeric strut 54, or the recess 362 to form a flexible space which is similar to a leaf spring in order to cushion the axial compression between two vertebrae.

Further, as shown in U.S. Pat. No. 7,001,433, this patent uses the combination of the ball bearing body 13 which is flexible and the bearing body 17 to cushion the axial compression between two vertebrae.

As shown in US publication number US2006/0064169A1, this patent reveals the diversified use of combination or application mode of the spring 112 and piston assembly 110 which is used to be the structure to cushion the compression.

As shown in U.S. Pat. No. 7,001,433 and US publication number US2006/0293753A1, these patents use polymeric material to wrap all kinds of cushion elements such as spring, damping spring, and structure beam to make the artificial intervertebral disc have a more powerful cushion structure.

Furthermore, as shown in US publication number US2006/0178744A1, this patent uses the core 216 to achieve the purpose of torsion activity, wherein this patent uses the elastic element of inner sandwich structure of the plate 230 and 232 to cushion the axial compression between two vertebrae.

Finally, as shown in US publication number US2006/0282165A1, this patent uses structure which is similar to a leaf spring only to cushion the axial compression between two vertebrae but not to provide the function of torsion activity between two vertebrae.

The above prior arts all have the function to cushion the axial compression between two vertebrae, but fewer of them can simulate the displacement capability of the real intervertebral disc under the condition of being compressed, wherein only the elastomer knitted by fiber can achieve forgoing goal; however, the elastomer knitted by fiber can not provide a wide range of torsion activity angle as the other artificial intervertebral disc shown above. The conventional artificial intervertebral disc can not provide both the displacement capability of the real intervertebral disc under the condition of being compressed and a wide range of torsion activity angle at the same time, which is a defect that all current techniques extremely want to improve.

Furthermore, the majority of the aforementioned technologies are only conceptual designs used to describe the function of how to cushion the compression; when it comes to product-oriented structure, it may be totally different from the conventional structure so that the cost of aforementioned artificial intervertebral disc will be too high and then result in economic stress of the patient. If disregarding the compatibility between aforementioned artificial intervertebral disc and the modern surgical instrument, the conceptual design may easily lead to a technical gaps and difficulties during practical surgical operation. The above is a tough question that the design of the artificial intervertebral disc has to overcome.

### SUMMARY OF THE INVENTION

Due to the defect of foregoing artificial intervertebral discs, the present invention provides a displacement leaf spring and an artificial intervertebral disc containing the same. When using the displacement leaf spring, the artificial intervertebral disc can have a wide range of torsion activity angle and capability to cushion the axial compression, just like the real one.

In order to achieve above purpose, the present invention comprising the displacement leaf spring, and the displacement leaf spring comprising an upper plate, an inclined plate, and a lower plate, wherein the upper plate having a first breach, and the inclined plate having a first connecting side which connects to one side of the first breach. There is an angle between the inclined plate and the upper plate. The inclined plate further having a second breach, and the lower plate having a second connecting side which connects to one side of the second breach. There is an angle between the lower plate and the inclined plate, and the lower plate parallels the upper plate. In addition to the displacement leaf spring, the artificial intervertebral disc further comprising a top plate, a bottom plate, a core structure, and a socket structure, wherein the top plate and the bottom plate connect to the bottom of the upper vertebral body and the top of the lower vertebral body respectively; the form of the core structure fits in the socket structure, and the core structure and the socket structure are installed between the top and the bottom plate. Above shall be the basic structure of the present invention.

By the above-mentioned technical solution, the present invention compared to known technology has at least the following advantages:
1. The use of the conventional artificial intervertebral discs composed of elastic buffer makes the activity angle of the upper and the lower vertebral body restricted. The core structure and the socket structure of the present invention are installed between the top plate and the bottom plate, and then the top plate and the bottom plate connect to the bottom of the upper vertebral body and the top of the lower vertebral body respectively, which makes the artificial intervertebral disc have a wide range of activity angle.
2. The conventional artificial intervertebral disc composed of elastomer which can be compressed in irregular or vertical direction such as spring, piston, and elastic buffer to cushion the axial compression. The use of the displacement leaf spring in the present invention is to cushion the axial compression of the artificial intervertebral disc. When the displacement leaf spring is compressed in the vertical direction, it absorbs the compression, which is just like the same function as the real intervertebral disc absorbing the compression; after that, said function results in lateral displacement.
3. The use of the displacement leaf spring and the artificial intervertebral disc in the present invention make the present invention have a wide range of activity angle and simulate the function of the real intervertebral disc to absorb the compression and then result in displacement.
4. Considering the compatibility between the artificial intervertebral disc of the present invention and the conventional artificial intervertebral disc, the present invention is made to fit the structure of conventional artificial intervertebral disc as far as possible, so that the present invention can make use of the known surgical instruments and procedures to avoid the trouble and risk of surgical operations, which provides patients with significant usage security and improves the effect of operations.

### BRIEF DESCRIPTION OF THE DRAWINGS

FIG. 1 is an external appearance schematic drawing of the displacement leaf spring of the present invention.

FIG. 2 is a cross-sectional view of the present invention of FIG. 1 taken along line A-A.

FIG. 3-1 is the first schematic side view of the displacement leaf spring of the present invention.

FIG. 3-2 is the second schematic side view of the displacement leaf spring of the present invention.

FIG. 4-1 is the first decomposition chart of the artificial intervertebral disc equipped with the displacement leaf spring of the present invention.

FIG. 4-2 is the second decomposition chart of the artificial intervertebral disc equipped with the displacement leaf spring of the present invention.

FIG. 5-1 is the first side view of the artificial intervertebral disc equipped with the displacement leaf spring of the present invention.

FIG. 5-2 is the second side view of the artificial intervertebral disc equipped with the displacement leaf spring of the present invention.

FIG. 5-3 is the first front view of the artificial intervertebral disc equipped with the displacement leaf spring of the present invention.

FIG. 5-4 is the second front view of the artificial intervertebral disc equipped with the displacement leaf spring of the present invention.

FIG. 6 is another decomposition chart of the artificial intervertebral disc equipped with the displacement leaf spring of the present invention.

FIG. 7 is another cross-sectional view of the artificial intervertebral disc equipped with the displacement leaf spring of the present invention.

FIG. 8 is another decomposition chart of the artificial intervertebral disc equipped with the displacement leaf spring of the present invention.

FIG. 9 is another cross-sectional view of the artificial intervertebral disc equipped with the displacement leaf spring of the present invention.

FIG. 10 is another external appearance schematic drawing of the displacement leaf spring of the present invention.

FIG. 11 is a cross-sectional view of the present invention of FIG. 10 taken along line B-B.

FIG. 12 is another external appearance schematic drawing of the displacement leaf spring of the present invention.

FIG. 13 is a cross-sectional view of the present invention of FIG. 12 taken along line C-C.

### DETAILED DESCRIPTION OF THE INVENTION

These and other aspects and embodiments will be described in further detail below, with reference to the drawing figures.

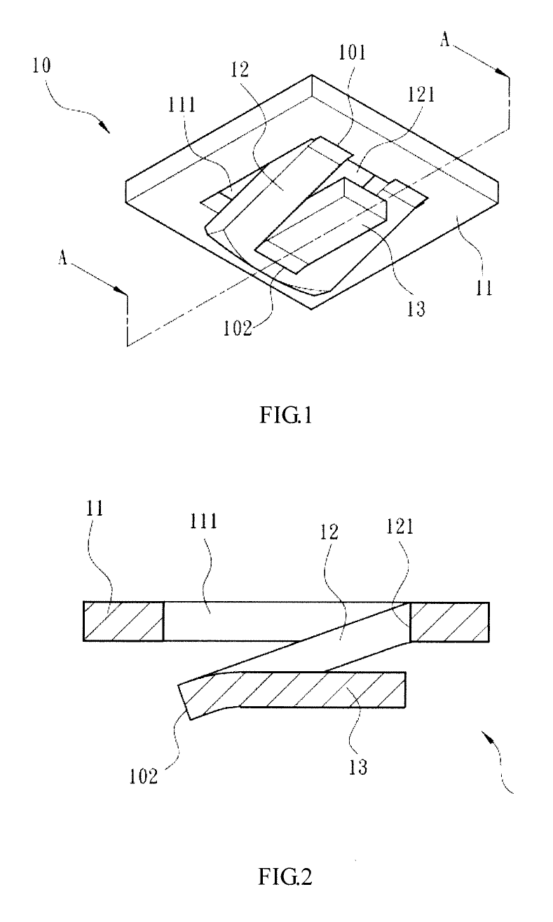

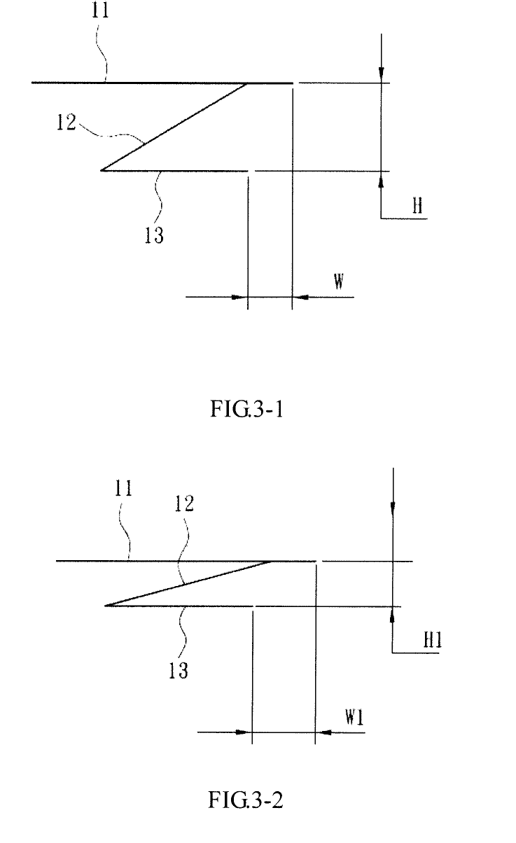

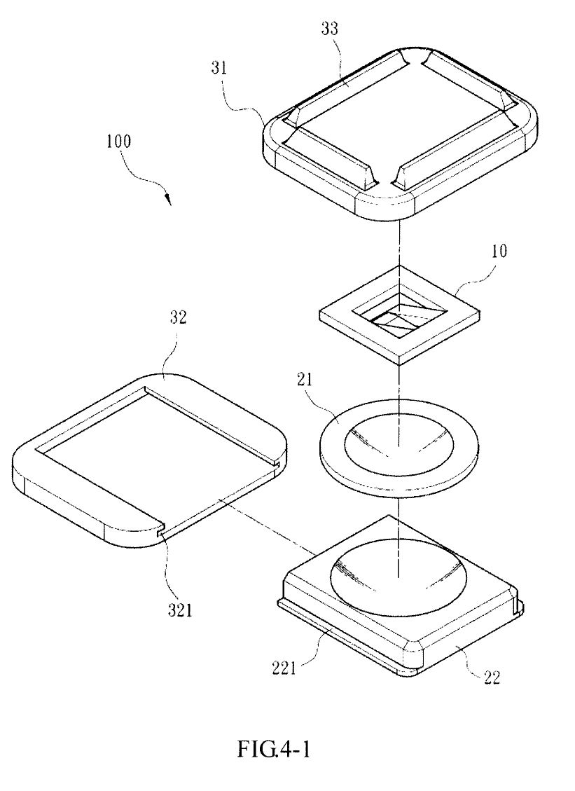

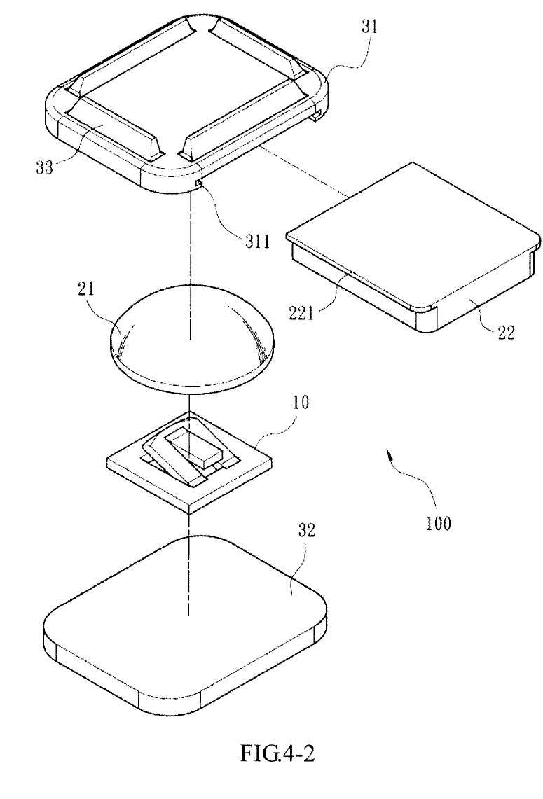

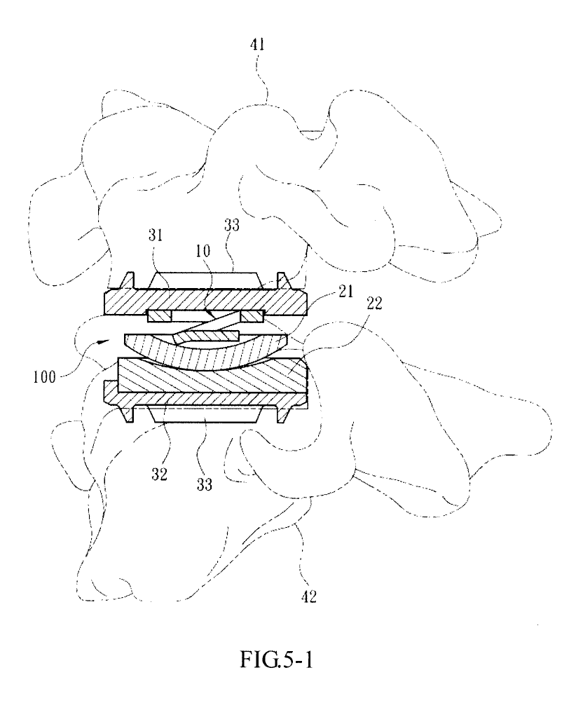

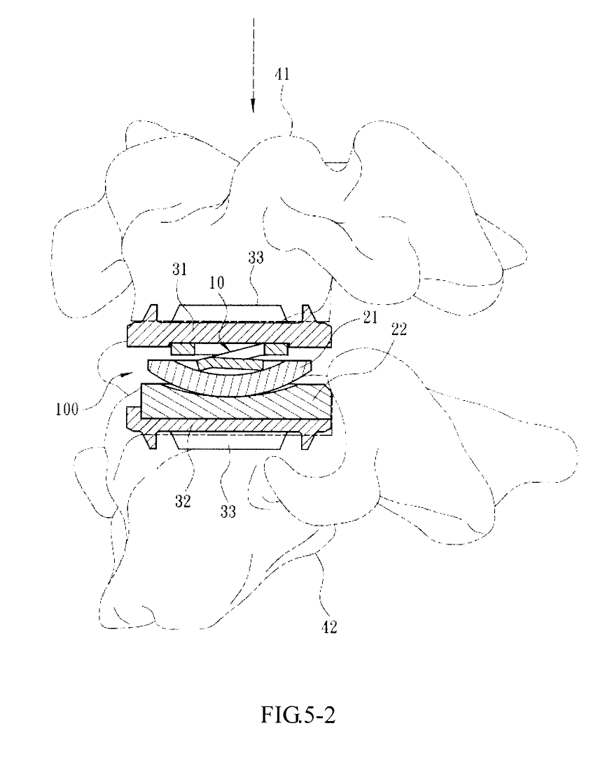

In the first place, there is a structure of the displacement leaf spring 10 of the present invention with reference to FIG. 1 and FIG. 2, the thing which shown in the figure is only a better embodiment of the present invention and is not intended to be limited to one or the other of above embodiment, wherein the displacement leaf spring 10 comprises an upper plate 11, an inclined plate 12, and a lower plate 13; the upper plate has a first breach 111, the inclined plate 12 has a first connecting side 101 which connects to one side of the first breach 111, and there is an angle between the inclined plate 12 and the upper plate 11; the inclined plate 12 further has a second breach 121, the lower plate 13 has a second connecting side 102 which connects to one side of the second breach 121, and there is an angle between the lower plate 13 and the inclined plate 12 to make the lower plate 13 parallel the upper plate 11. Above shall be the basic structure of the displacement leaf spring 10 in the present invention, wherein the upper plate 11, the inclined plate 12, and the lower plate 13 are formed integrally and composed of restoring resilient material so that the displacement leaf spring 10 can cushion and absorb the compression by resilient capability between the upper plate 11 and the lower plate 13.

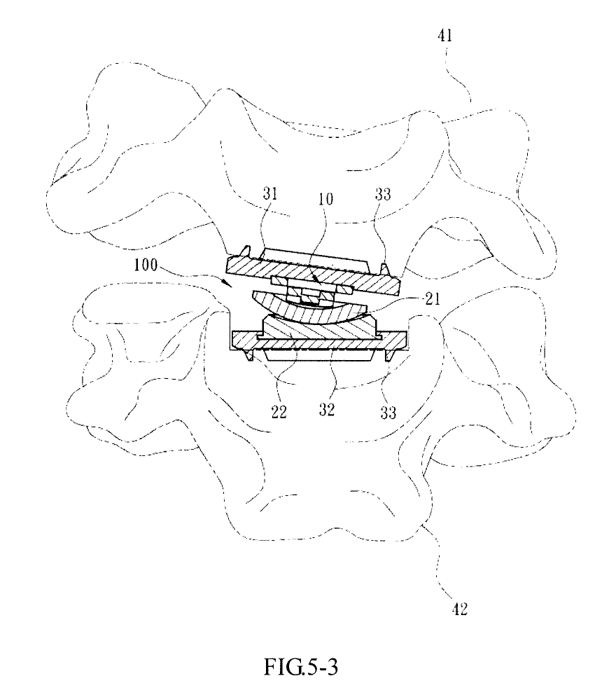

In order to illustrate the displacement capability of the displacement leaf spring 10 under the condition of being compressed, please refer to FIG. 3-1 and FIG. 3-2; FIG. 3-1 and FIG. 3-2 show the schematic side view of the displacement leaf spring 10, as shown in the figure, the displacement leaf spring 10 has a Z-shaped appearance. As shown in FIG. 3-1, the relative vertical length between the upper plate 11 and the lower plate 13 is marked as H, and the relative horizontal length between the upper plate 11 and the lower plate 13 is marked as W. When there is axial force imposed on the upper plate 11 and the lower plate 13, the displacement leaf spring 10 will be compressed and then deformed as shown in FIG. 3-2; in this condition, the relative vertical length between the upper plate 11 and the lower plate 13 is marked as H1, and the relative horizontal length between the upper plate 11 and the lower plate 13 is marked as W1. As shown in FIG. 3-1 and FIG. 3-2, the vertical length H is longer than the vertical length H1, and the horizontal length W1 is longer than the horizontal length W. It can be saw clearly that the vertical length between the upper plate 11 and the lower plate 13 will be reduced, and the horizontal length between the upper plate 11 and the lower plate 13 will be changed as well when the displacement leaf spring 10 under the condition of being compressed; that is to say, besides the vertical displacement capability of the displacement leaf spring 10 under the condition of being compressed, there will be a kind of horizontal displacement (from horizontal length W to horizontal length W1) capability provided.

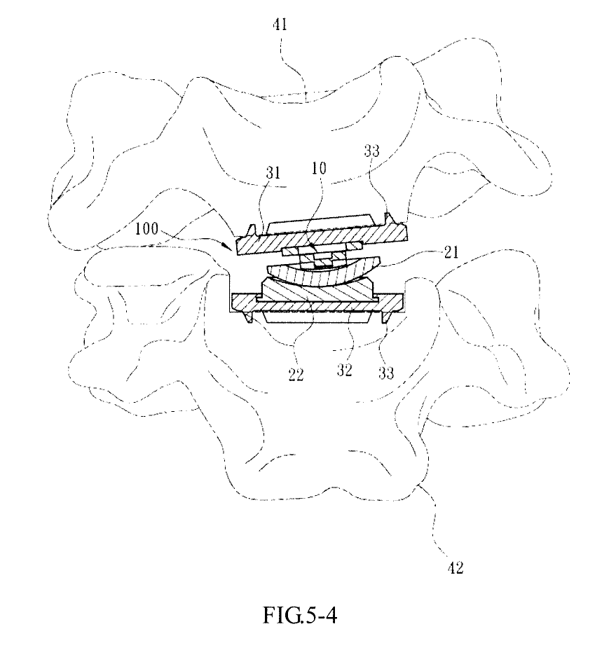

The use of said displacement leaf spring 10 can cushion the compression and provide a kind of horizontal displacement property; moreover, the use of said displacement leaf spring 10 can apply to an artificial intervertebral disc 100. FIG. 4-1 shows a decomposition chart of the artificial intervertebral disc 100 equipped with displacement leaf spring 10, wherein said decomposition chart of artificial intervertebral disc comprises a top plate 31 and a bottom plate 32, the top plate 31 and the bottom plate 32 connect to the bottom of the upper vertebral body 41 (shown in FIG. 5-3) and the top of the lower vertebral body 42 respectively; the artificial intervertebral disc 100 further comprises a core structure 21 and a socket structure 22, wherein the form of the core structure 21 fits in the socket structure 22, and the core structure 21 and the socket structure 22 are installed between the top plate 31 and the bottom plate 32.

The core structure 21 and the socket structure 22 of the present invention are installed between the top plate 31 and the bottom plate 32, and then the top plate 31 and the bottom plate 32 connect to the bottom of the upper vertebral body 41 and the top of the lower vertebral body 42 respectively, which makes the artificial intervertebral disc 100 have a wide range of activity angle. The displacement leaf spring 10 provides a characteristic to cushion and absorb the axial compression. The artificial intervertebral disc 100 further comprises at least one mounting component 33 which is between the top plate 31 and the bottom of the upper vertebral body 41 and between the bottom plate 32 and the top of the lower vertebral body 42; the artificial intervertebral disc 100 can be firmly fixed to the upper vertebral body 41 and the lower vertebral body 42 by the use of the mounting component 33 with reference to the embodiment side view 1 and 2 of artificial intervertebral disc 100 equipped with the displacement leaf spring 10 as shown in FIG. 5-1 and FIG. 5-2.

The condition of the displacement leaf spring 10 between the upper vertebral body 41 and the lower vertebral body 42 which changes from not under compression to under compression provides a buffer force for the artificial intervertebral disc 100 to cushion the axial compression. As the embodiment front view 1 and 2 of artificial intervertebral disc 100 equipped with the displacement leaf spring 10 as shown in FIG. 5-3 and FIG. 5-4, the condition of relative movement swing between the upper vertebral body 41 and the lower vertebral body 42 by relative movement between the core structure 21 and the socket structure 22 is used to simulate the displacement characteristic and the torsion angle as the real intervertebral disc under compression, which makes the present invention much closer to the primary intervertebral disc and increases the safety and comfort of the patient.

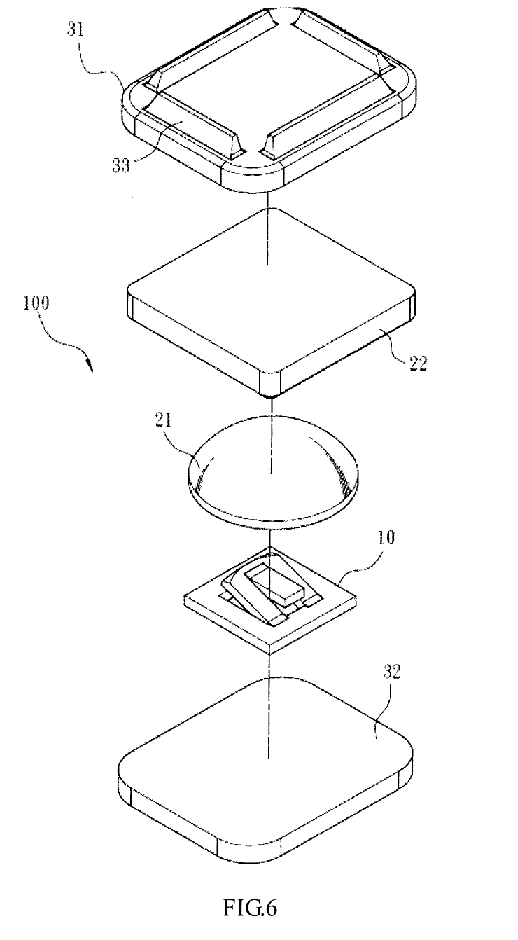

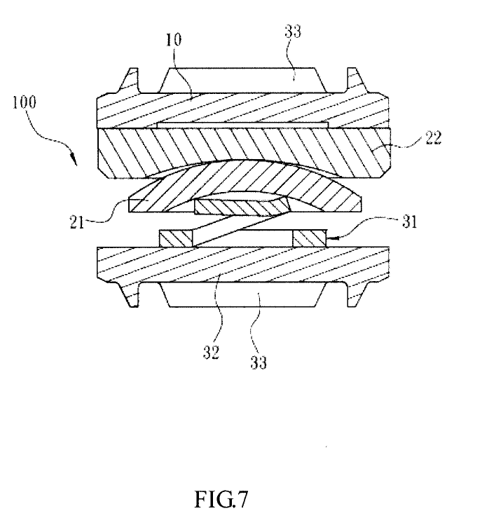

The displacement leaf spring 10 may be installed between the core structure 21 and the top plate 31 as shown in FIG. 4-1 or between the core structure 21 and the bottom plate 32 as shown in FIG. 4-2, FIG. 6, FIG. 7, wherein the artificial intervertebral disc 100 equipped with the displacement leaf spring 10 can be not only composed of independent components separated from each other but composed of a variety of assembly selecting from each component, such as when the displacement leaf spring 10 is installed between the core structure 21 and the top plate 31, the socket structure 22 and the bottom plate 32 are formed integrally; when the displacement leaf spring 10 is installed between the core structure 21 and the bottom plate 32, the socket structure 22 and the top plate 31 are formed integrally.

In addition, besides the method of forming integrally between the socket structure 22 and the top plate 31 or between the socket structure 22 and the bottom plate 32, and the connection method between the socket structure 22 and the top plate 31 or between the socket structure 22 and the bottom plate 32 can make use of tenon as shown in FIG. 4-1, wherein when the displacement leaf spring 10 is installed between the core structure 21 and the top plate 31, the bottom plate 32 has two fixed chutes 321; the socket structure 22 has two fixed convex portions 221 corresponding to the fixed chute 321 so that the socket structure 22 slides into the fixed chute 321 and then combines with the bottom plate 32 by the fixed convex portion 221. As shown in FIG. 4-2, when the displacement leaf spring 10 is installed between the core structure 21 and the bottom plate 32, the top plate 31 has two fixed chutes 311; the socket structure 22 has two fixed convex portions 221 corresponding to the fixed chute 311 so that the socket structure 22 slides into the fixed chute 311 and then combines with the top plate 31 by the fixed convex portion 221. The above shall be another condition of connection method between the socket structure 22 and the top plate 31 or between the socket structure 22 and the bottom plate 32.

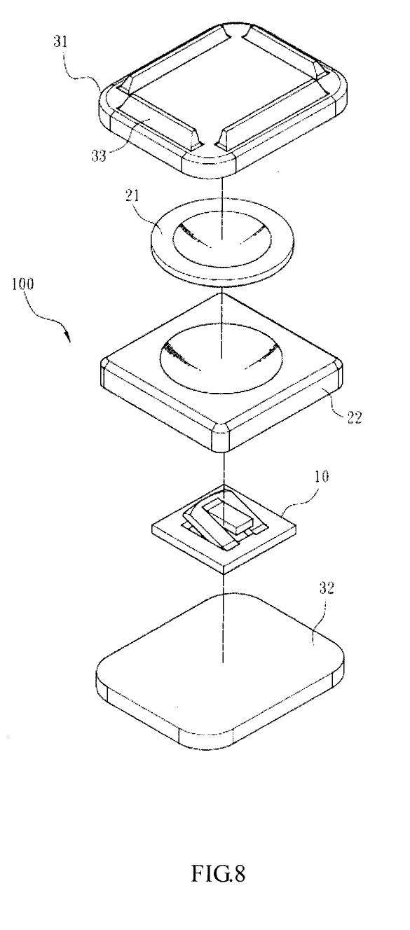

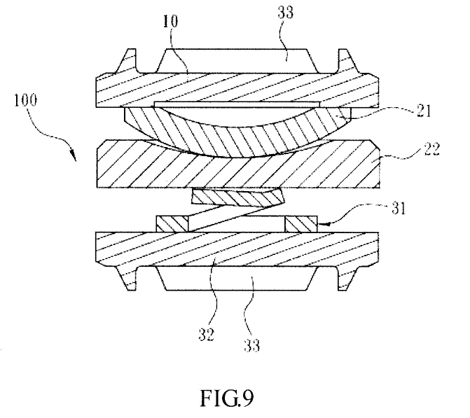

Another component disposition mode of the artificial intervertebral disc 100 as shown in FIG. 8 and FIG. 9, the displacement leaf spring 10 may be installed between the socket structure 22 and the bottom plate 32 but not limit to the displacement leaf spring 10, the displacement leaf spring 10 may also be installed between the socket structure 22 and the top plate 31, and the artificial intervertebral disc 100 equipped with the displacement leaf spring 10 can be not only composed of independent components separated from each other but composed of a variety of assembly selecting from each component, such as when the displacement leaf spring 10 is installed between the socket structure 22 and the top plate 31, the core structure 21 and the bottom plate 32 are formed integrally; when the displacement leaf spring 10 is installed between the socket structure 22 and the bottom plate 32, the core structure 21 and the top plate 31 are formed integrally.

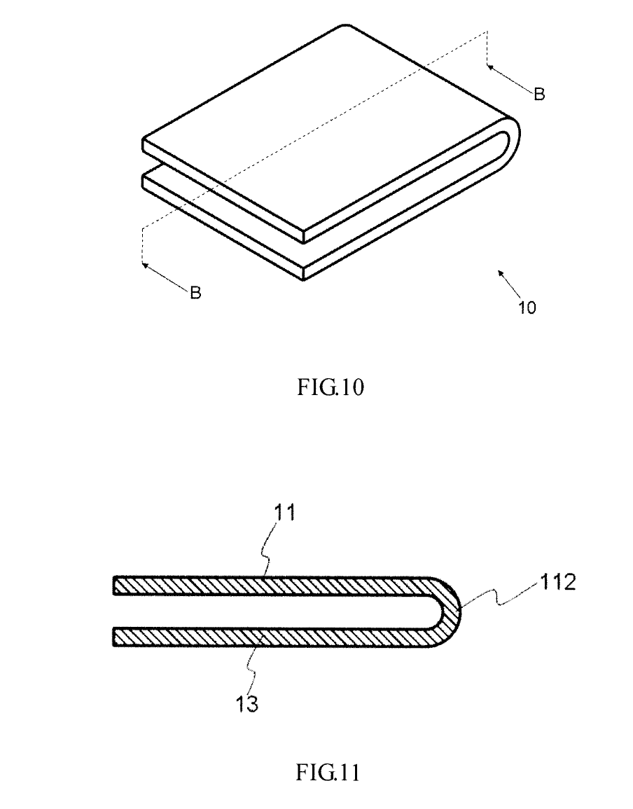

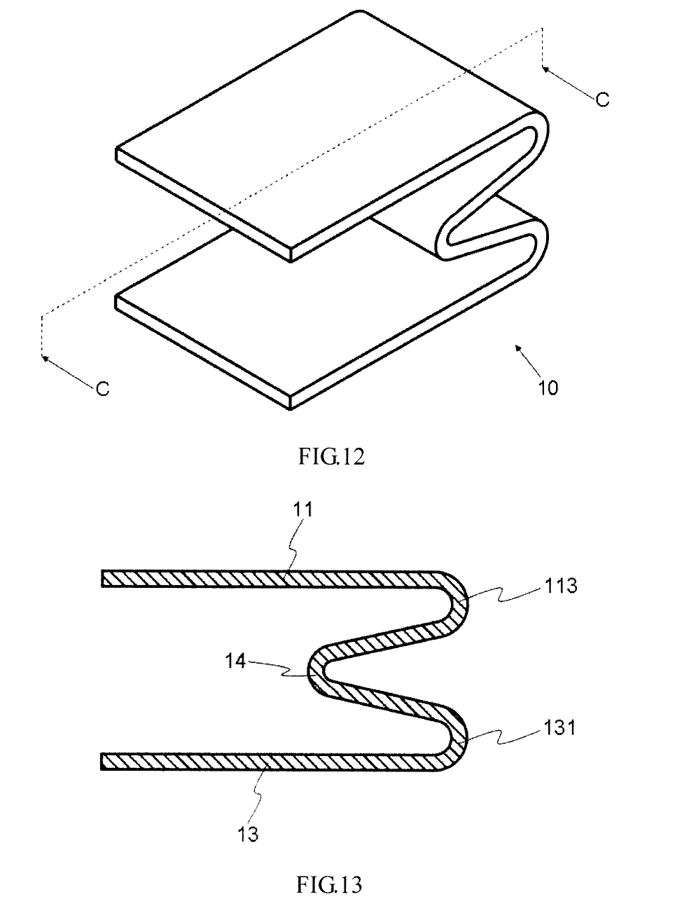

The best embodiment of the displacement leaf spring 10 of the present invention has been shown above, but there is another embodiment of the displacement leaf spring 10 which can cushion the compression and provide a kind of horizontal displacement with reference to FIG. 10 and FIG. 11, wherein the displacement leaf spring 10 comprises an upper plate 11 and a lower plate 13; the upper plate 11 has a bending portion 112, and the lower plate 13 is able to connect with the bending portion 112 of the upper plate 11, which makes the lower plate 13 parallel the upper plate 11. The above is another basic structure of the displacement leaf spring 10, wherein the displacement leaf spring 10 is formed integrally and composed of restoring resilient material so that the displacement leaf spring 10 can cushion and absorb the compression by resilient capability between the upper plate 11 and the lower plate 13; Another embodiment of the displacement leaf spring 10, please refer to FIG. 12 and FIG. 13, wherein the displacement leaf spring 10 comprising an upper plate 11, a lower plate 13, and a third bending portion 14; the upper plate 11 has a first bending portion 113 shaped by extending from the edge of the upper plate 11 and then bending inward, the lower plate 13 has a second bending portion 131 shaped by extending from the edge of the lower plate 13 and then bending inward, and the third bending portion 14 extending from two extremities of itself and then connecting to the first bending portion 113 and second bending portion 131 respectively, which makes the upper plate 11 parallel the lower plate 13. The above is another basic structure of the displacement leaf spring 10, wherein the displacement leaf spring 10 is formed integrally and composed of restoring resilient material so that the displacement leaf spring 10 can cushion and absorb the compression by resilient capability between the upper plate 11 and the lower plate 13. The displacement capability of the displacement leaf spring 10 under the condition of being compressed in above-mentioned two embodiments are equal to the best embodiment, which does not have to give unnecessary details.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A displacement leaf spring for artificial intervertebral disc comprising:
an upper plate having a first breach;
an inclined plate having a first connecting side which connects to one side of the first breach, there is an angle between the inclined plate and the upper plate, the inclined plate further having a second breach; and
a lower plate having a second connecting side which connects to one side of the second breach, there is an angle between the lower plate and the inclined plate, and the lower plate parallels the upper plate.

2. A displacement leaf spring for artificial intervertebral disc according to claims 1, wherein the displacement leaf spring is formed integrally and composed of restoring resilient material.

3. An artificial intervertebral disc equipped with the displacement leaf spring comprising:
a top plate and a bottom plate connect to the bottom of the upper vertebral body and the top of the lower vertebral body respectively;
a core structure and a socket structure, the form of the core structure fits in the socket structure, and the core structure and socket structure are installed between the top plate and the bottom plate; and
a displacement leaf spring may be installed between the top plate and the core structure, between the top plate and the socket structure, between the bottom plate and the core structure, or between the bottom plate and the socket structure.

4. An artificial intervertebral disc equipped with the displacement leaf spring according to claim 3, wherein the displacement leaf spring comprising an upper plate, an inclined plate, and a lower plate; the upper plate has a first breach, the inclined plate has a first connecting side which connects to one side of the first breach, and there is an angle between the inclined plate and the upper plate; the inclined plate further has a second breach, the lower plate has a second connecting side which connects to one side of the second breach, there is an angle between the lower plate and the inclined plate, and the lower plate parallels the upper plate.

5. An artificial intervertebral disc equipped with the displacement leaf spring according to claim 3, wherein the displacement leaf spring comprising an upper plate and a lower plate; the upper plate has a bending portion, the lower plate is able to connect with the bending portion of the upper plate, which makes the lower plate parallel the upper plate.

6. An artificial intervertebral disc equipped with the displacement leaf spring according to claim 3, wherein the displacement leaf spring comprising an upper plate, a lower plate, and a third bending portion; the upper plate has a first bending portion shaped by extending from the edge of the upper plate and then bending inward, the lower plate has a second bending portion shaped by extending from the edge of the lower plate and then bending inward, the third bending portion extending from two extremities of itself and then connecting to the first and second bending portion respectively, which makes the upper plate parallel the lower plate.

7. An artificial intervertebral disc equipped with the displacement leaf spring according to claim 4, 5, or 6, wherein the displacement leaf spring is formed integrally and composed of restoring resilient material.

8. An artificial intervertebral disc equipped with the displacement leaf spring according to claim 3, wherein the artificial intervertebral disc further comprising at least one mounting component which is between the top plate and the bottom of the upper vertebral body and between the bottom plate and the top of the lower vertebral body.

9. An artificial intervertebral disc equipped with the displacement leaf spring according to claim 3, wherein the socket structure and the bottom plate are formed integrally when the displacement leaf spring is installed between the core structure and the top plate; the socket structure and the top plate are formed integrally when the displacement leaf spring is installed between the core structure and the bottom plate; the core structure and the bottom plate are formed integrally when the displacement leaf spring is installed between the socket structure and the top plate; the core structure and the top plate are formed integrally when the displacement leaf spring is installed between the socket structure and the bottom plate.

10. An artificial intervertebral disc equipped with the displacement leaf spring according to claim 9, wherein as the displacement leaf spring is installed between the core structure and the top plate, the bottom plate has two fixed chutes, and the socket structure has two fixed convex portions corresponding to the fixed chute so that the socket structure slides into the fixed chute and then combines with the bottom plate by the fixed convex portion; as the displacement leaf spring is installed between the core structure and the bottom plate, the top plate has two fixed chutes, and the socket structure has two fixed convex portions corresponding to the fixed chute so that the socket structure slides into the fixed chute and then combines with the top plate by the fixed convex portion.
